(19) United States
(12) Patent Application Publication
(10) Pub. No.: US 2017/0052136 A1
(43) Pub. Date: Feb. 23, 2017

(54) ELECTRODE AND METHOD FOR DETECTING ANALYTE USING SAME

(71) Applicant: LG ELECTRONICS INC., Seoul (KR)

(72) Inventors: Keumcheol KWAK, Seoul (KR); Woongkee MIN, Seoul (KR); Karyakin ARKADY, Moscow (RU)

(21) Appl. No.: 15/301,101

(22) PCT Filed: Feb. 16, 2015
(86) PCT No.: PCT/KR2015/001564

§ 371 Date: Sep. 30, 2016

(30) Foreign Application Priority Data
Apr. 2, 2014 (KR) 10-2014-0039514

(51) Int. Cl. G01N 27/12; G01N 33/487

(52) U.S. Cl. CPC G01N 27/126; G01N 33/48707

(57) ABSTRACT

The method for detecting an analyte of the present invention comprises the steps of: coating an electrode with at least one of an aniline polymer formed by substituting hydrogen of a monomer with a boronic acid, followed by electropolymerization, a thiopene polymer, a pyrrole polymer, a carbon nanotube and graphene; exposing the electrode to an analyte that is present in a solution or air; and measuring impedance generated from the electrode exposed to the analyte. The present invention forms the aniline polymer by electropolymerization of an aniline monomer which is formed by substituting hydrogen with a boronic acid in the step of coating the electrode, and utilizes the electrode as a biosensor after coating the electrode with the aniline polymer. Further, in the step of measuring impedance, the presence/absence of an analyte is detected from a change in conductivity.

### TECHNICAL FIELD

The present invention relates to a method for detecting microorganisms such as mold.

### BACKGROUND ART

Sugars in the form of independent bodies (free) or glycoconjugates (glycoproteins and glycolipids) are important biological molecules in various technology fields. Accordingly, it is an important problem in the technology fields to detect the presence and absence of sugars. Glucose sensors now routinely used to detect the presence and absence of sugars are based on electrochemical methods of detecting the glucose oxidized by enzyme and the redox active species produced in this process.

The biosensor field has been significantly developed by methods of using non-physiological electron acceptors and methods of using the direct electron transfer conducted between the active site of enzyme and the electrode. Nevertheless, in general, the low stability of enzymes and the sensitive change in activity of the enzyme associated with various factors which cannot be controlled are known as the limitations of the biosensor. For this reason, in the current research areas, sugar sensors which do not use an enzyme have been very actively developed.

Among them, one approach is a method based on the direct electrocatalytic oxidation of glucose. This method has been developed by using metal nanoparticles as a catalyst and using a carbon nanotube or graphene material as a catalyst support, but the systems reported to date still have a problem in that the activity of the catalyst is insufficient under the physiological environment.

Biosensors, which do not use an enzyme, use lectins that naturally make specific bondings with sugars in a natural way, and are configured in the form that concanavalin A is usually used. However, a more successful approach than the method is to introduce a boronic acid receptor capable of selectively binding a compound having a 1,2 or 1,3-diol functional group, and the compound having a 1,2 or 1,3-diol functional group is a common constituent element of sugars. The advantages of using a synthetic receptor are low costs, high stability, and the ability to relatively easily change the structure in order to apply the synthetic receptor to optimal sensing properties. In order to apply these results to a biosensor, a reaction of forming esters needs to be accompanied simultaneously by a signaling process which causes a change in optical (absorption, fluorescence, or holographic) or electrical properties.

There are prior art documents that configure an optical sensor based on the aforementioned approach.

Methods tried in U.S. Pat. Nos. 6,011,984 and 5,512,246 are methods of detecting glucose by using dyes attached to boronic acid groups and using a fluorescence method. The patents use the fact that when a boronic acid including a functional dye binds to sugar, the dye is affected by characteristics. Electrochemical sensors are actuated by an aqueous electroactive compound or a film fixed on the surface of an electrode.

The sensors of the first type disclosed in U.S. Pat. No. 6,011,984 most frequently used ferrocenyl boronic acid, and the ferrocenyl boronic acid generates a change in oxidation reduction potential caused by a complexation reaction in the presence of hydroxyl groups. In addition, the sensors of the second type disclosed in U.S. Pat. No. 5,512,246 used a conductive polymer modified with a boronic acid functional group. The sensors of the second type were carried out by a method of forming a boronic acid single layer on a gold electrode, or a method of forming a polymer layer including a boronic acid functional group on an electrode.

The approach to an electrochemical glucose sensor is reported in U.S. Pat. No. 6,797,152, and the patent used a method of binding an aniline boronic acid polymer to glucose, and generating signals depending on the concentration of glucose. The signal to be analyzed changes an open-circuit potential. The patent failed to suggest an experimental evidence in spite of the conductivity mentioned in the claim. Furthermore, the concentrations of glucose and fructose, which can be maximally detected, were about 40 mM.

When all the prior art sensors previously mentioned are reviewed, the sensors were described from one or more different viewpoints in terms of electrical polymerization conditions of an aniline boronic acid polymer, the form of analytical signal, a detectable concentration range, and the like.

### DISCLOSURE OF THE INVENTION

Therefore, an object of the present invention is to provide a method for detecting an analyte commonly having a cis-diol group, such as sugars, hydroxylic acids, polyhydroxyl acids, and polyhydroxyl aldehydes, and an electrode capable of detecting an analyte having a cis-diol group.

Another object of the present invention is to provide a method capable of identifying the presence and absence of an analyte by the five senses via detection of the electrical signals.

To achieve these and other advantages and in accordance with the purpose of the present invention, as embodied and broadly described herein, there is provided a method for detecting an analyte according to an example of the present invention including: coating an electrode with at least one of an aniline polymer, a thiophene polymer, a pyrrole polymer, carbon nanotube, and graphene, which are formed by substituting hydrogen of each monomer with boronic acid and subjecting the resulting monomer to electropolymerization; exposing the electrode to an analyte present in a solution or in the air; and measuring an impedance generated from the electrode exposed to the analyte.

According to an example related to the present invention, the coating of the electrode includes: reacting at least one of an aniline monomer, a thiophene monomer, a pyrrole monomer, a monomer of carbon nanotube, and a monomer of graphene with phenyl boronic acid to substitute hydrogen of the monomer with boronic acid; subjecting the monomer in which hydrogen is substituted with boronic acid to electropolymerization at a predetermined acid concentration and under a predetermined anodic switching potential condition; and coating the electrode with the polymer formed by electropolymerization.

According to another example related to the present invention, it is possible to determine the presence and absence of the analyte from a change in conductivity.

According to still another example related to the present invention, the analyte may include at least one of glucose, fructose, lactic acid, galactose, sialic acid, and microorganisms.

According to yet another example related to the present invention, the aniline polymer includes a 3-aminophenylboronic acid polymer formed by substituting hydrogen of an aniline monomer with boronic acid and subjecting the resulting monomer to electropolymerization, and the a 3-aminophenylboronic acid polymer may be formed by subjecting a 3-aminophenylboronic acid monomer to electropolymerization under conditions of maintaining a sulfuric acid (H₂SCO₄) concentration of 0.2 M or less and an anodic switching potential of 0.9 V or less compared to an Ag/AgCl reference electrode.

Further, the present invention discloses an electrode including a cis-diol group receptor in order to realize the aforementioned problem. In an electrode provided in a sensor which detects an analyte having a cis-diol group by measuring an impedance, the electrode includes a cis-diol group receptor, and the cis-diol group receptor is formed by being coated with at least one of an aniline polymer, a thiophene polymer, a pyrrole polymer, carbon nanotube, and graphene, which are formed by substituting hydrogen of each monomer with boronic acid and subjecting the resulting monomer to electropolymerization.

According to an example related to the present invention, the cis-diol group receptor may be configured so as to be reacted with a sugar having a cis-diol group in a carbon ring or bacteria.

According to the present invention as described above, it is possible to provide an electrochemical detection method of a material including sugar and cis-diol such as hydroxyl acid. The present invention uses a product obtained by coating an electrode with an aniline polymer formed by substituting hydrogen of an aniline monomer with boronic acid and subjecting the resulting monomer to electropolymerization as a biosensor, and the present invention may determine the presence and absence of an analyte by measuring the impedance.

In addition, the present invention may provide a user with information on the presence and absence of an analyte by transmitting a result of a change in conductivity shown as a result of detection to the user.

### BRIEF DESCRIPTION OF THE DRAWINGS

FIG. 1 is a molecular structure illustrating an example of an analyte to be detected by using a method for detecting the analyte proposed by the present invention;

FIG. 2 is a flowchart of the method for detecting the analyte related to an example of the present invention;

FIGS. 3a and 3b are a molecular structure of an aniline monomer and an aniline polymer, respectively;

FIG. 4 is a molecular structure of phenyl boronic acid;

FIGS. 5a and 5b are a molecular structure of an aniline monomer in which hydrogen is substituted with boronic acid and an aniline polymer, respectively;

FIG. 6 is a graph for describing a condition of forming an aniline polymer by subjecting an aniline monomer in which hydrogen is substituted with boronic acid to electropolymerization;

FIG. 7 is a graph of the results of experiments for demonstrating the accuracy and effectiveness of the analyte detection method of the present invention;

FIG. 8 is an equivalent circuit used in the experiment of FIG. 7;

FIG. 9 is a graph illustrating the results of the detection of glucose by using the method for detecting an analyte according to the present invention at each concentration;

FIG. 10 is a graph illustrating the results of the detection of fructose by using the method for detecting an analyte according to the present invention at each concentration;

FIG. 11 is a graph illustrating the results of the detection of lactate by using the method for detecting an analyte according to the present invention at each concentration;

FIG. 12 is a graph illustrating the results of the detection of galactose by using the method for detecting an analyte according to the present invention at each concentration; and

FIG. 13 is a block configuration view showing the sensor of the present invention, which detects the analyte.

### MODES FOR CARRYING OUT THE PREFERRED EMBODIMENTS

Reference will now be made in detail to the preferred embodiments of the present invention, examples of which are illustrated in the accompanying drawings. It will also be apparent to those skilled in the art that various modifications and variations can be made in the present invention without departing from the spirit or scope of the invention. Thus, it is intended that the present invention cover modifications and variations of this invention provided they come within the scope of the appended claims and their equivalents.

Description will now be given in detail of a drain device and a refrigerator having the same according to an embodiment, with reference to the accompanying drawings.

Hereinafter, an electrode related to the present invention and a method for detecting analyte using the same will be described in more detail with reference to the drawings. In the present specification, like reference numbers are used to designate like constituents even though they are in different Examples, and the description thereof will be replaced with the initial description. Singular expressions used in the present specification include plural expressions unless they have definitely opposite meanings in the context.

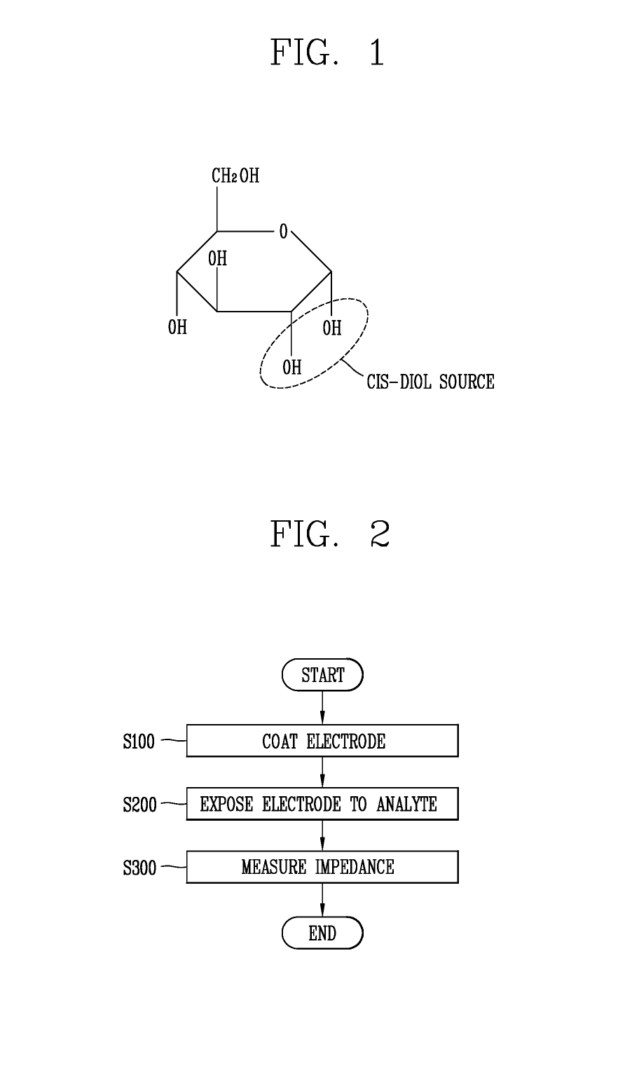

FIG. 1 illustrates a molecular structure illustrating an example of an analyte to be detected by using a method for detecting the analyte proposed by the present invention.

The molecule illustrated in FIG. 1 is glucopyranose, which is one of sugars. Glucopyranose is a material having cis-diol (cis-diols source) in a carbon ring as indicated by a dotted line. Sugars, hydroxyl acids, polyhydroxyl acids, and polyhydroxyl aldehydes to be detected in the present invention are targeted to materials commonly having cis-diol in a carbon ring. For example, mold also has cis-diol in a carbon ring, and thus may be an analyte of the present invention. The analyte may include at least one of glucose, fructose, lactic acid, galactose, sialic acid, and microorganisms.

Hereinafter, the detection method of the present invention for detecting an analyte having cis-diol in a carbon ring will be described.

FIG. 2 is a flowchart of the method for detecting the analyte related to an example of the present invention.

The method for detecting an analyte includes coating an electrode (S100), exposing the electrode to an analyte (S200), and measuring an impedance (S300).

First, the coating of the electrode (S100) is a process of preparing the electrode which will be used in the detection of the analyte. The electrode in the present invention is coated with at least one of an aniline polymer (electropolymerized boronic acid substituted polyaniline), a thiophene polymer (electropolymerized boronic acid substituted polythiophene), a pyrrole polymer (electropolymerized boronic acid substituted polypyrrole), carbon nanotube (electropolymerized boronic acid substituted carbon nanotube), and graphene (electropolymerized boronic acid substituted graphene), which are formed by substituting hydrogen of each monomer with boronic acid and subjecting the resulting monomer to electropolymerization.

The electrode may be provided in a sensor which detects an analyte having a cis-diol group by measuring an impedance. The electrode formed by the aforementioned process includes a cis-diol group receptor. For example, the cis-diol group receptor is formed by being coated with at least one of an aniline polymer, a thiophene polymer, a pyrrole polymer, carbon nanotube, and graphene, which are formed by substituting hydrogen of each monomer with boronic acid and subjecting the resulting monomer to electropolymerization. The cis-diol group receptor is configured so as to be reacted with a sugar having a cis-diol group in a carbon ring or bacteria.

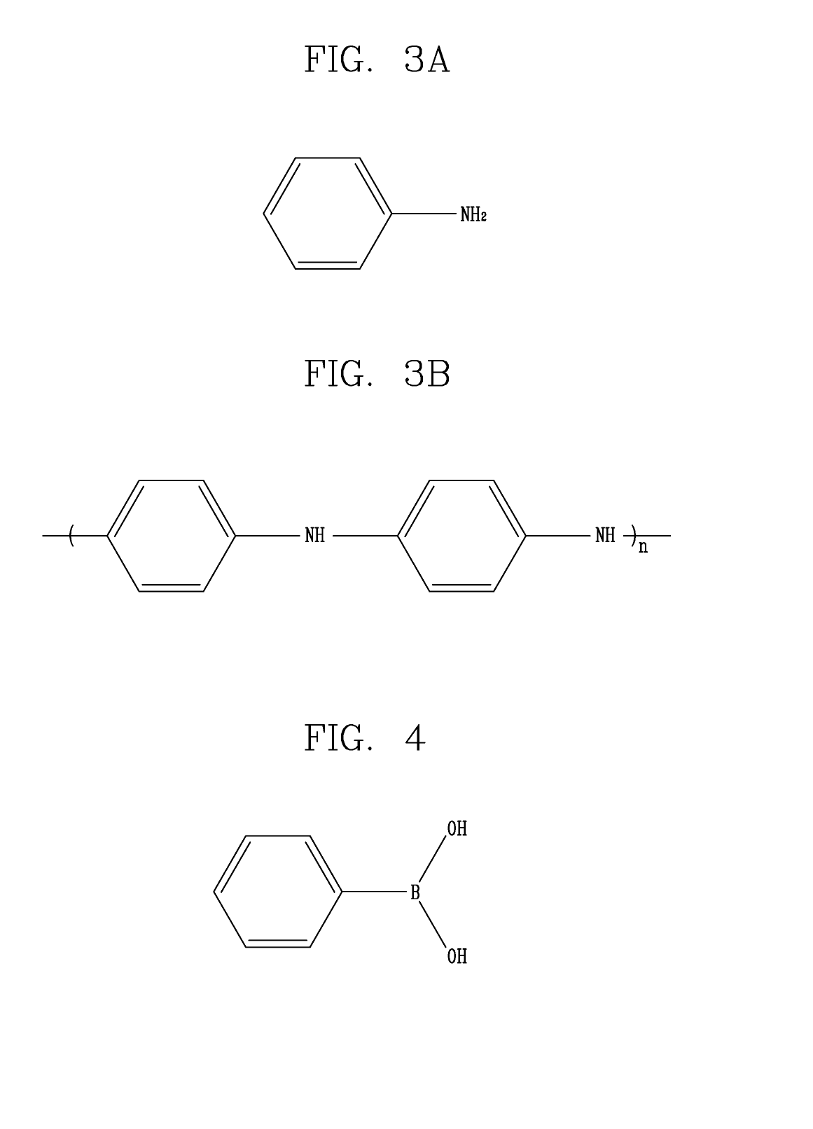

Hereinafter, the coating of the electrode (S100) will be described in more detail with reference to FIGS. 3a to 7. The description will be described below for an aniline polymer, but may also be applied to the aforementioned thiophene polymer, pyrrole polymer, carbon nanotube, and graphene.

FIGS. 3a and 3b illustrate a molecular structure of an aniline monomer and an aniline polymer, respectively.

More specifically, FIG. 3a illustrate an aniline monomer before hydrogen is substituted with boronic acid. Moreover, FIG. 3b illustrates an aniline polymer formed by subjecting the aniline monomer to electropolymerization before substituting hydrogen with boronic acid.

In the present invention, the aniline monomer or aniline polymer is not coated as it is onto an electrode. In the present invention, an electrode is coated with an aniline polymer (electropolymerized boronic acid substituted polyaniline) formed by first substituting hydrogen of an aniline monomer with boronic acid and subjecting the aniline monomer in which hydrogen is substituted with boronic acid (boronic acid substituted polyaniline) to electropolymerization.

FIG. 4 illustrates a molecular structure of phenyl boronic acid.

Phenyl boronic acid is reacted with the aniline monomer. Phenyl boronic acid provides the aniline monomer with boronic acid, and hydrogen of the aniline monomer is substituted with boronic acid. Boronic acid acts as a receptor of cis-diol.

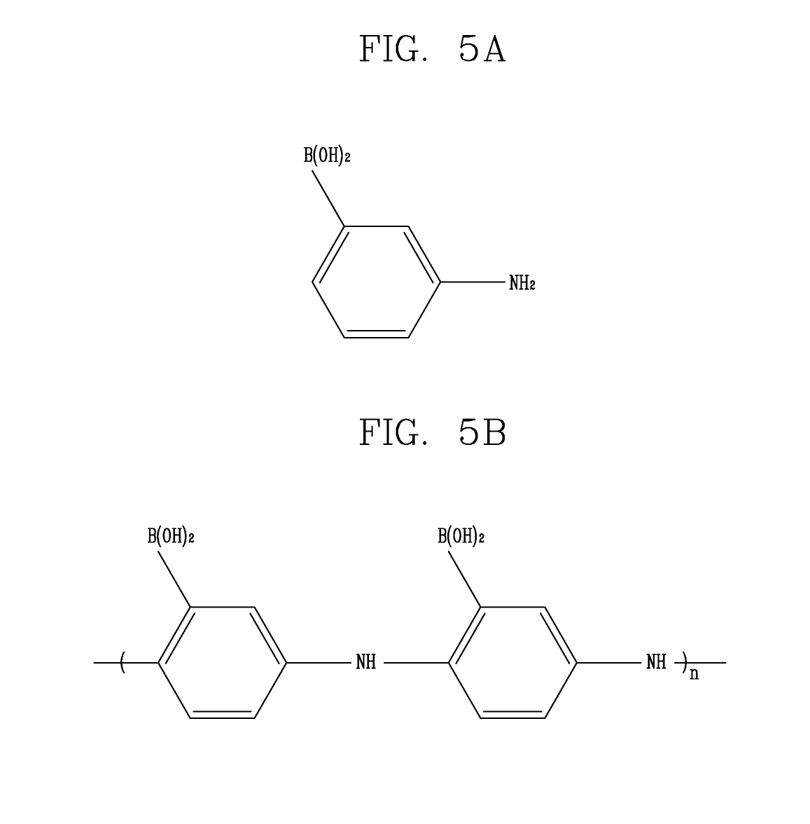

FIG. 5a is an aniline monomer in which hydrogen is substituted with boronic acid, and FIG. 5b illustrates a molecular structure of an aniline polymer formed by subjecting the aniline monomer of FIG. 5a to electropolymerization.

FIG. 5a is a 3-aminophenylboronic acid monomer formed by reacting the aniline monomer illustrated in FIG. 3a with the phenyl boronic acid illustrated in FIG. 4. Moreover, FIG. 5b is a 3-aminophenylboronic acid polymer formed by subjecting the 3-aminophenylboronic acid monomer illustrated in FIG. 5a to electropolymerization.

In the present invention, an electrode is coated with an aniline polymer (electropolymerized boronic acid substituted polyaniline) formed by subjecting the aniline monomer in which hydrogen is substituted with boronic acid to electropolymerization.

Depending on the environment of subjecting the aniline monomer in which hydrogen is substituted with boronic acid to electropolymerization, the conductivity of the electrode may vary. Hereinafter, an electropolymerization atmosphere suitable for the present invention will be described.

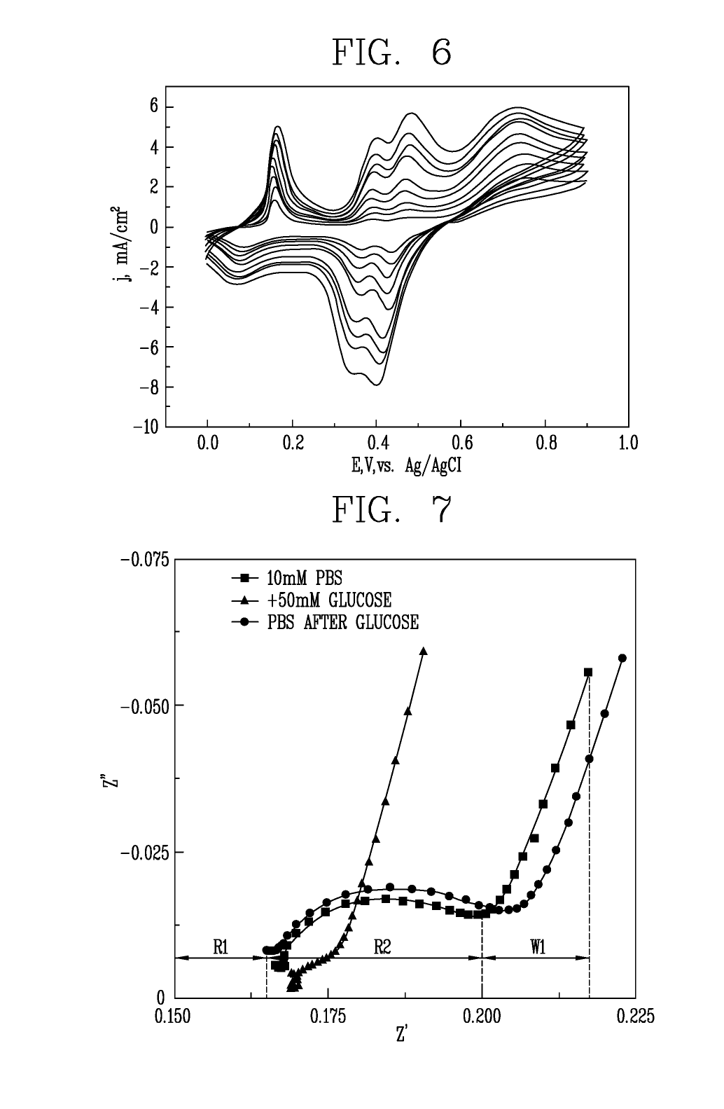

FIG. 6 is a graph for describing a condition of forming an aniline polymer by subjecting an aniline monomer in which hydrogen is substituted with boronic acid to electropolymerization.

There are two conditions for electropolymerization required for the present invention. One is a concentration of acid, and the other is an anodic switching potential.

First, the concentration of acid will be described.

For the aniline monomer in which hydrogen is substituted with boronic acid, as the concentration of acid is increased, the growth rate of the polymer is increased. In contrast, the aniline monomer in which hydrogen is substituted with boronic acid, such as 3-aminophenylboronic acid, is the opposite to the description described above. Accordingly, as the concentration of acid of the aniline monomer in which hydrogen is substituted with boronic acid, such as 3-aminophenylboronic acid, is increased, the growth rate of the polymer is increased.

A conductive polymer is formed by electropolymerization, and for example, an aniline polymer is formed. As the pH of the electropolymerization environment is increased, the conductivity range of the polymerized aniline polymer moves toward the negative potential. This is because the aniline polymer polymerized at a high pH environment has low conductivity. Accordingly, it is not preferred that electropolymerization is carried out in a range where the concentration of acid is extremely low. In the present invention, a suitable concentration of acid should be lower than 0.2 M of H2_{S}O₄.

Next, an anodic switching potential will be described.

The anodic switching potential determines the formation of anionic radicals, which require polymerization. However, polymers formed at relatively high anodic switching potential are known to have a relatively low conductivity. In the present invention, it is preferred that the anodic switching potential is maintained at 0.9 V or less compared to an Ag/AgCl reference electrode, and at the anodic switching potential in this range, a stable polymer is formed.

Under an environment of a relatively low sulfuric acid concentration and a low anodic switching potential, the electropolymerized aniline polymer is an element which is important for determining the performance of the method for detecting the analyte proposed by the present invention.

When the graph illustrated in FIG. 6 is reviewed, it can be confirmed that electropolymerization is carried out while maintaining the anodic switching potential at 0.9 V or more compared to the Ag/AgCl reference electrode. The electropolymerization environment of FIG. 6 includes 0.04 M of 3-aminophenylboronic acid (3-APBA), 0.2 of NaF, 0.125 M of sulfuric acid (H₂SO₄), and 40 mV/s of the scanning rate (sweep rate) of the electropolymerization.

When an electrode is coated with the polymerized aniline polymer according to the conditions of the electropolymerization required for the present invention, the preparation of the electrode required for detection of the analyte is completed.

Again, referring to FIG. 2, the electrode completely coated is exposed to an analyte present in a solution or in the air (S200).

When the analyte is present in the air, the electrode is exposed to the air. When the analyte is present in the solution, the electrode is introduced into the solution.

Finally, an impedance generated from the electrode exposed to the analyte is measured (S300).

As described in FIG. 1, the analyte includes cis-diols. Moreover, the electrode is coated with an aniline polymer in which hydrogen is substituted with boronic acid. A composite of hydroxyl groups with boronic acid residues (complexation of polyols with boronic residues) redistributes the electron density in a polymer ring. Thus, the conductivity observed in the impedance spectrum is increased.

Accordingly, the present invention may detect the presence and absence of an analyte when an impedance generated from an electrode is measured. The present invention may detect hydroxyl groups (polyols) up to a concentration of about 350 mM.

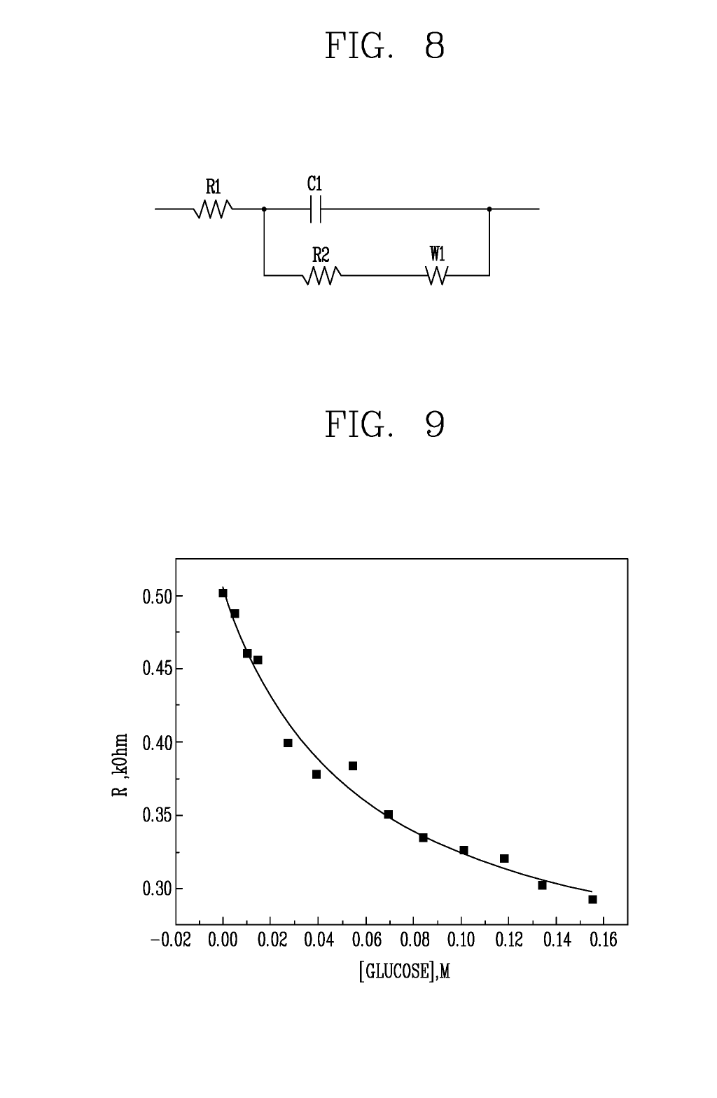

FIG. 7 is a graph of the results of experiments for demonstrating the accuracy and effectiveness of the analyte detection method of the present invention. Moreover, FIG. 8 is an equivalent circuit used in the experiment of FIG. 7.

In the experiment, a phosphate buffer saline (PBS) with a pH value of 7 was used in the solution, and glucose was introduced as the analyte into the phosphate buffer saline.

In the graph, (a), (b), and (c) are the results obtained by sequentially measuring the impedance. (a) is a result obtained by introducing an electrode into the phosphate buffer saline before adding glucose thereto and measuring the impedance. (b) is a result obtained by adding glucose thereto and measuring the impedance. (b) is a result obtained by again removing glucose and measuring the impedance.

In the graph, all of (a), (b), and (c) may be classified into R1 Region, R2 Region, and W1 Region. However, in FIGS. 7, R1, R2, and W1 were indicated only for (a) in order to prevent the drawing from being complicated. Referring to the symbols indicated in (a), the left side of R2 Region in which a semicircle is drawn corresponds to R1 Region, and the right side of R2 Region corresponds to W1 Region. The principle described above is also applied to (b) and (c). Also in (b) and (c), the region in which a semicircle is drawn corresponds to R2, the left side of R2 corresponds to R1, and the right side of R2 corresponds to W1. The region to be carefully reviewed is R2 Region. When R2 Region is reviewed, it can be confirmed that the impedance or conductivity changes, and through this, it can be confirmed whether the analyte is present.

When (a) is compared with (b), it can be confirmed that as glucose is added to the solution, R2 Region is decreased. Accordingly, the conductivity of the electrode is more increased under an environment in which the analyte is present than under an environment in which the analyte is not present. When (b) is compared with (c), it can be confirmed that as the analyte is removed from the solution, R2 Region is again increased. Accordingly, the conductivity of the electrode is more decreased under an environment in which the analyte is not present than under an environment in which the analyte is present.

Through the graphs (a), (b), and (c) in which the impedance is measured, the present invention may determine the presence and absence of the analyte from a change in conductivity. Moreover, when the determined result is formed as signals, and converted into a form which a person can recognize by the five senses, the present invention may be utilized as a sensor capable of determining the presence and absence of the analyte. For example, when the detection results using the present invention are converted into visual and auditory signals, it is possible to provide a user with information capable of confirming the presence and absence of the analyte.

Hereinafter, specific examples of the present invention will be described.

In the experiment, electropolymerization of the aniline polymer was first carried out, and subsequently, the effectiveness of the present invention was verified by comparing the results before and after adding glucose thereto. Finally, the detection was tried by applying the present invention to glucose, fructose, lactate, and galactose.

The materials and apparatuses used in the following Examples are as follows.

3-aminophenylboronic acid (3-APBA), D-glucose, fructose, lactic acid, and galactose, all manufactured by Aldrich Chemical Co., Inc., were purchased, and the purchased products were used as they were at the time of purchase. As all the inorganic salts required for the experiment, products manufactured by Reachim were purchased and used as they were at the time of purchase. A sample was prepared by hermetically sealing glass-phase carbon electrodes (diameter 1.8 mm) with Teflon and grinding the electrodes.

The electrochemical experiments were carried out by using Micro 3 products manufactured by PalmSens and Autolab. The impedance was measured by using a 1255 Frequency Response Analyser product manufactured by Solartron Schlumberger. The experiment was carried out by using an Ag/AgCl reference electrode and three electrodes prepared as a counter electrode of platinum wire. All the measurements were carried out under the atmospheric conditions (25° C.) without a rigorous regulation for the temperature overload.

### EXAMPLE 1

Example 1 is an electropolymerization of 3-APBA.

The electropolymerization was carried out with a composition of 40 mM of 3-aminophenylboronic acid, 0.125 M of sulfuric acid (H₂SO₄), and 0.2 M of NaF. The potential of the working electrode was scanned in a range of 0 to 0.9 V compared to an Ag/AgCl reference electrode, the scanning rate was 40 mV/s, and the potential was scanned 10 times. The electropolymerization of 3-APBA coated onto a glass phase carbon electrode is illustrated in FIG. 6. In order to prove the quality of the polymer film coated onto the electrode, cyclic voltammograms were obtained from a supporting electrolyte (0.1 M of KCl immersed in 0.1 M of HCl).

### EXAMPLE 2

Subsequently, the effectiveness of the present invention was verified by comparing the results before and after adding glucose thereto.

An electrode coated with an aniline polymer (hereinafter, referred to as a biosensor) was prepared in the order as described in Example 1. Moreover, the biosensor was immersed in a phosphate buffer saline with a pH value of 7.0 for 4 hours. Subsequently, the measurement was carried out at a perturbation amplitude of 3.5 mV in a frequency range of 20 kHz to 20 Hz (FIG. 7(a)). The potential was maintained at −50 mV while the measurement was carried out. And then, glucose at 50 mM was introduced from an original concentrated solution. The spectra of the phosphate buffer saline after glucose was added (FIG. 7(b)) and a pure phosphate buffer saline containing no glucose (FIG. 7(c)) were reported. FIG. 7 illustrates the changes in impedance spectra. Accordingly, in the present invention, it is possible to draw a calibration graph for glucose by using an appropriate frequency.

The effectiveness of the present invention is replaced with those described in FIG. 7.

### EXAMPLE 3-1

The conductivities of glucose at different concentrations were measured by using the present invention.

FIG. 9 is a graph illustrating the results of the detection of the glucose by using the method for detecting an analyte according to the present invention at each concentration.

The biosensor was prepared in the order as described in Example 1. Moreover, the biosensor was immersed in a phosphate buffer saline with a pH value of 7.0 for 4 hours. Subsequently, the measurement was carried out at a perturbation amplitude of 3.5 mV in a frequency range of 20 kHz to 20 Hz. The potential was maintained at −50 mV while the measurement was carried out. And then, glucose was introduced from an original concentrated solution. Glucose was added thereto, and then the spectrum was reported at each time. FIG. 7 illustrates the changes in impedance spectra. The obtained spectrum was simulated in the Randles equivalent circuit model illustrated in FIG. 8. The R2 values are conductivity characteristics of the polymer-electrode system. The addition of glucose increased the conductivity of the polymer-electrode system, and accordingly, the R2 parameters were decreased. The calibration graph for glucose is illustrated in FIG. 9.

### EXAMPLE 3-2

The conductivities of fructose at different concentrations were measured by using the present invention.

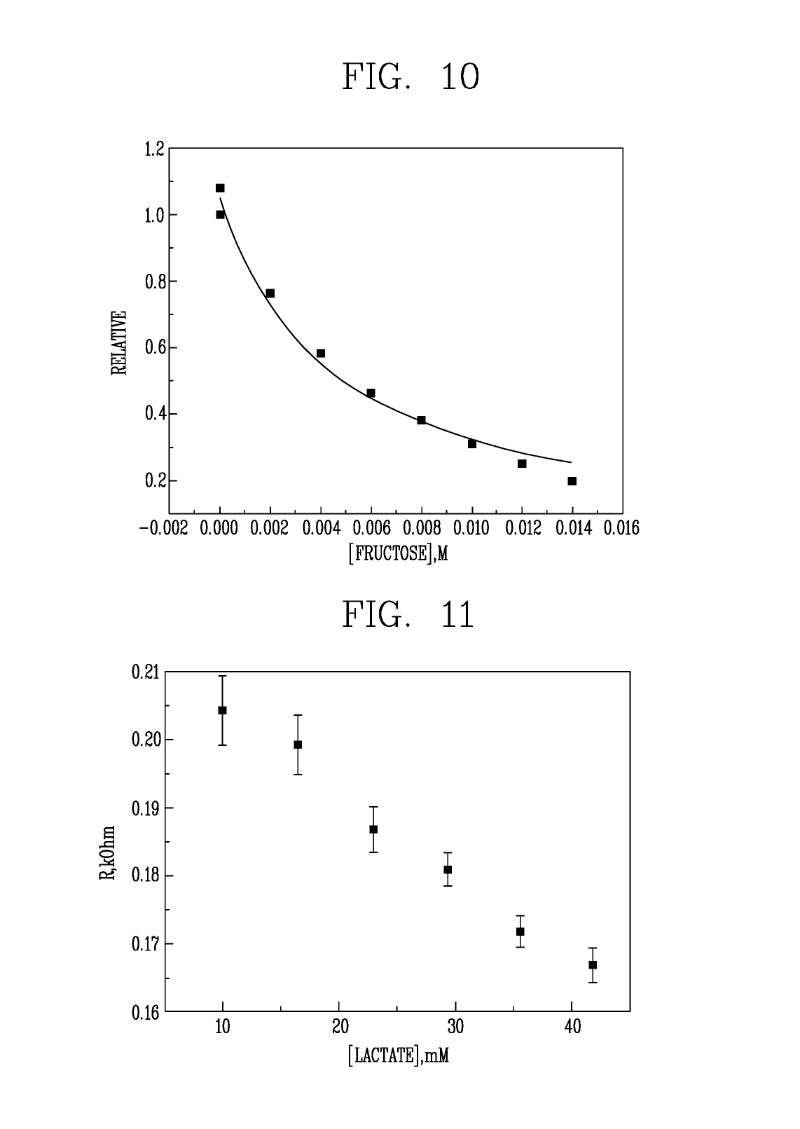

FIG. 10 is a graph illustrating the results of the detection of fructose by using the method for detecting an analyte according to the present invention at each concentration.

The biosensor was prepared in the order as described in Example 1. Moreover, the biosensor was immersed in a phosphate buffer saline with a pH value of 7.0 for 4 hours. Subsequently, the measurement was carried out at a perturbation amplitude of 3.5 mV in a frequency range of 20 kHz to 20 Hz. The potential was maintained at −50 mV while the measurement was carried out. And then, fructose was introduced from an original concentrated solution. Fructose was added thereto, and then the spectrum was reported at each time. The obtained spectrum was simulated in the Randles equivalent circuit model illustrated in FIG. 8. The addition of glucose increased the conductivity of the polymer-electrode system, and accordingly, the R2 parameters were decreased. The calibration graph for glucose is illustrated in FIG. 10.

### EXAMPLE 3-3

The conductivities of lactate at different concentrations were measured by using the present invention.

FIG. 11 is a graph illustrating the results of the detection of lactate by using the method for detecting an analyte according to the present invention at each concentration.

The biosensor was prepared in the order as described in Example 1. Moreover, the biosensor was immersed in a phosphate buffer saline with a pH value of 7.0 for 4 hours. Subsequently, the measurement was carried out at a perturbation amplitude of 3.5 mV in a frequency range of 20 kHz to 20 Hz. The potential was maintained at −50 mV while the measurement was carried out. And then, lactate was introduced from an original concentrated solution. Lactate was added thereto, and then the spectrum was reported at each time. The obtained spectrum was simulated in the Randles equivalent circuit model illustrated in FIG. 8. The addition of lactate increased the conductivity of the polymer-electrode system, and accordingly, the R2 parameters were decreased. The calibration graph for lactate is illustrated in FIG. 11.

### EXAMPLE 3-4

The conductivities of galactose at different concentrations were measured by using the present invention.

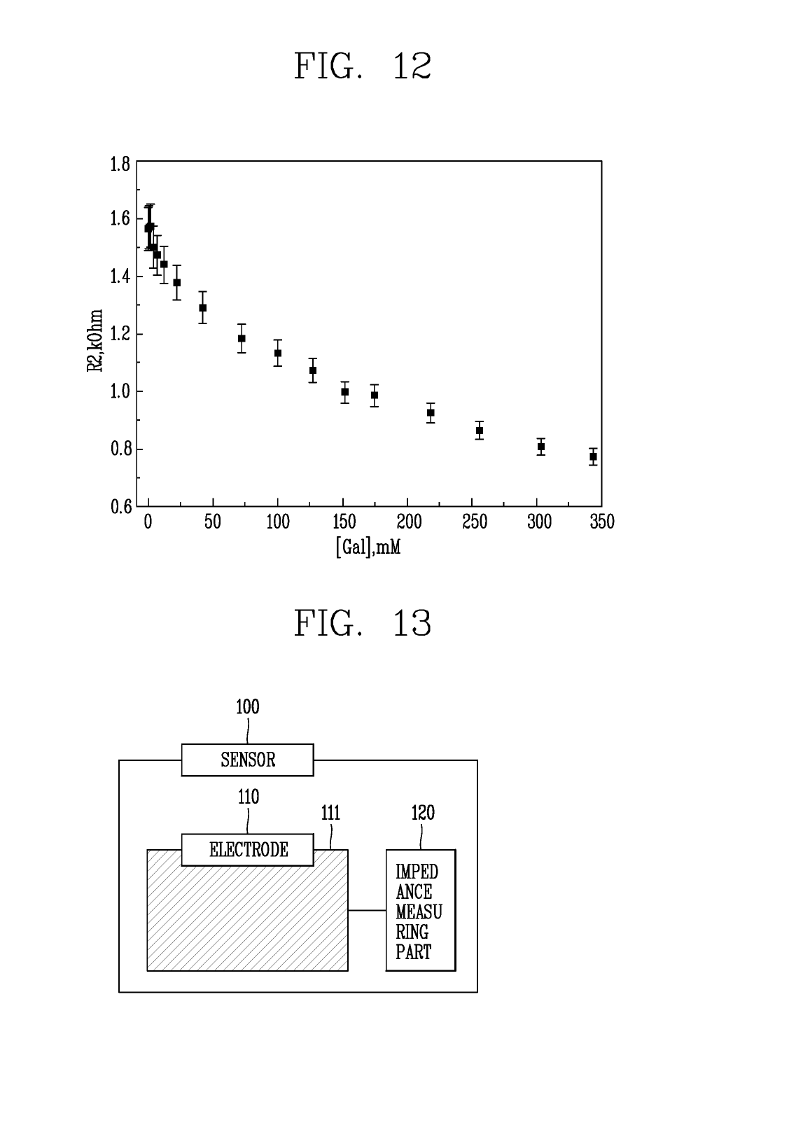

FIG. 12 is a graph illustrating the results of the detection of galactose by using the method for detecting an analyte according to the present invention at each concentration.

The biosensor was prepared in the order as described in Example 1. Moreover, the biosensor was immersed in a phosphate buffer saline with a pH value of 7.0 for 4 hours. Subsequently, the measurement was carried out at a perturbation amplitude of 3.5 mV in a frequency range of 20 kHz to 20 Hz. The potential was maintained at −50 mV while the measurement was carried out. And then, galactose was introduced from an original concentrated solution. Galactose was added thereto, and then the spectrum was reported at each time. The obtained spectrum was simulated in the Randles equivalent circuit model illustrated in FIG. 8. The addition of galactose increased the conductivity of the polymer-electrode system, and accordingly, the R2 parameters were decreased. The calibration graph for galactose is illustrated in FIG. 12.

As can be confirmed in FIGS. 9 to 12, as the concentration of the analyte (glucose, fructose, lactate, and galactose) is increased, the R2 parameters are decreased. This can be understood that as the concentration of the analyte is increased, the impedance is decreased, and the conductivity is increased. Accordingly, the present may detect the presence and concentration of the analyte from the increase in conductivity of the present invention.

Further, as can be confirmed in FIGS. 9 to 12, the present invention may be derived by quantifying the concentration of the analyte of the present invention into relatively exact values. Accordingly, the present invention may provide a user with information on the detection and concentration of the analyte. For example, when the present invention is used in a water purifier, the present invention may be used for use in demonstrating the performance of the water purifier because the degree of removing mold is quantified to provide a water purifier user with the quantified information.

FIG. 13 is a block configuration view showing a sensor 100 including the electrode 110 of the present invention, which detects the analyte.

The sensor 100 measures the impedance to detect an analyte having a cis-diol group. The sensor 100 may be used alone, and may also be used while being provided in a device such as a water purifier.

The sensor 100 includes the electrode 110, and a cis-diol group receptor 111 is provided in the electrode 110.

The cis-diol group receptor 111 is formed by being coated with at least one of an aniline polymer, a thiophene polymer, a pyrrole polymer, carbon nanotube, and graphene, which are formed by substituting hydrogen of each monomer with boronic acid and subjecting the resulting monomer to electropolymerization. The cis-diol group receptor 111 is configured so as to be reacted with a sugar having a cis-diol group in a carbon ring or bacteria, and the like.

The sensor includes an impedance measuring part 120, and the impedance measuring part 120 is configured so as to determine the presence and absence of the analyte from a change in conductivity of the electrode 110. Accordingly, the sensor 100 may sense the presence and absence of the analyte.

The electrode and the method for detecting an analyte using the same described above are not limited by the configurations and methods of the examples described above, but the examples may also be configured by selectively combining a whole or part of the examples, such that various modifications can be made.

### INDUSTRIAL APPLICABILITY

The present invention may be variously used in the industrial fields which require the detection of all the sugars having a cis-diol group or bacteria.

1. A method for detecting an analyte, the method comprising:
coating an electrode with at least one of an aniline polymer, a thiophene polymer, a pyrrole polymer, carbon nanotube, and graphene, which are formed by substituting hydrogen of each monomer with boronic acid, followed by electropolymerization;
exposing the electrode to an analyte present in a solution or in the air; and
measuring an impedance generated from the electrode exposed to the analyte.

2. The method of claim 1, wherein the coating of the electrode comprises:
reacting at least one of an aniline monomer, a thiophene monomer, a pyrrole monomer, a monomer of carbon nanotube, and a monomer of graphene with phenyl boronic acid to substitute hydrogen of the monomer with boronic acid;
subjecting the monomer in which hydrogen is substituted with boronic acid to electropolymerization at a predetermined acid concentration and under a predetermined anodic switching potential condition; and
coating the electrode with the polymer formed by electropolymerization.

3. The method of claim 1, wherein the analyte comprises at least one of glucose, fructose, lactic acid, galactose, sialic acid, and microorganisms.

4. The method of claim 1, wherein in the measuring of the impedance, a presence and absence of the analyte is determined from a change in conductivity.

5. The method of claim 1, wherein the aniline polymer comprises a 3-aminophenylboronic acid polymer formed by substituting hydrogen of an aniline monomer with boronic acid and subjecting the resulting monomer to electropolymerization, and
the a 3-aminophenylboronic acid polymer is formed by subjecting a 3-aminophenylboronic acid monomer to electropolymerization under conditions of maintaining a sulfuric acid (H₂SCO₄) concentration of 0.2 M or less and an anodic switching potential of 0.9 V or less compared to an Ag/AgCl reference electrode.

6. An electrode provided in a sensor which detects an analyte having a cis-diol group by measuring an impedance,
wherein the electrode comprises a cis-diol group receptor, and the cis-diol group receptor is formed by being coated with at least one of an aniline polymer, a thiophene polymer, a pyrrole polymer, carbon nanotube, and graphene, which are formed by substituting hydrogen of each monomer with boronic acid and subjecting the resulting monomer to electropolymerization.

7. The electrode of claim 6, wherein the cis-diol group receptor is configured so as to be reacted with a sugar having a cis-diol group in a carbon ring or bacteria.
